(19) United States
(12) Patent Application Publication
(10) Pub. No.: US 2024/0335381 A1
(43) Pub. Date: Oct. 10, 2024

(54) Transdermal Microemulsion Delivery Systems for Alcohol-Soluble Species Including Nonderivatized Hormones

(71) Applicant: Quicksilver Scientific, Inc., Louisville, CO (US)

(72) Inventors: Christopher W. Shade, Louisville, CO (US); Steven Tieu, Louisville, CO (US); James R Huey, Louisville, CO (US)

(73) Assignee: Quicksilver Scientific, Inc., Louisville, CO (US)

(21) Appl. No.: 18/629,522

(22) Filed: Apr. 8, 2024

Related U.S. Application Data
(63) Continuation of application No. PCT/US22/46677, filed on Oct. 14, 2022.
(60) Provisional application No. 63/272,328, filed on Oct. 27, 2021.

(51) Int. Cl. A61K 9/107; A61K 9/00; A61K 31/57; A61K 47/10; A61K 47/14; A61K 47/22; A61K 47/24; A61K 47/36; A61K 47/44; A61P 5/24

(52) U.S. Cl. CPC A61K 9/1075; A61K 9/0014; A61K 31/57; A61K 47/10; A61K 47/14; A61K 47/22; A61K 47/24; A61K 47/36; A61K 47/44; A61P 5/24

(57) ABSTRACT

Thickened microemulsions are described where hydrophobic liquid droplets are distributed in a continuous hydrophilic liquid phase. In relation to conventional oil-in-water (OIW) microemulsions, the described thickened microemulsions may be thought of as modified oil-in-water (MOIW) microemulsions, where both the “oil” and “water” phases of the microemulsion are modified. The oil phase droplets of the thickened MOIW microemulsion are modified with alcohol and an alcohol-lipid phase thickener and can solubilize alcohol-soluble species, including nonderivatized hormones. Preferably, the modified oil phase droplets of the thickened MOIW microemulsion directly solubilize nonderivatized hormones. The polar continuous “water” phase of the thickened MOIW microemulsion is modified with a continuous phase thickener. The modified oil phase droplets disperse into the modified polar continuous phase of the thickened MOIW microemulsion.

### RELATED APPLICATIONS

This application is a continuation of International Application No. PCT/US22/46677, filed Oct. 14, 2022, entitled “Transdermal Microemulsion Delivery Systems for Alcohol-Soluble Species Including Nonderivatized Hormones”, which claims the benefit of U.S. Provisional Application No. 63/272,328, entitled “Progesterone Topical”, filed Oct. 27, 2021, both of which are incorporated by reference in its entirety.

### BACKGROUND

Hormone replacement therapy (HRT) is used extensively for treating hormone deficiencies due to aging or pathological effects on the endocrine system. HRT also may be used to change secondary sexual characteristics of transgender people. Commonly used HRT hormones to combat menopause or andropause include testosterone, DiHydroTestosterone (DHT), DeHydroEpiAndrosterone (DHEA), 7-keto DHEA, progesterone, pregnenolone, estrogen, estradiol, estriol, androstenedione, and androstenediol.

Other than by injection or implant, the delivery of nonderivatized hormones to mammalian organisms can be difficult or unwise from a liver toxicity perspective. If delivered orally, conventional delivery systems often result in extensive metabolism of nonderivatized hormone in the liver, which may modify or render the hormone ineffective, and cause undesirable liver stress.

For example, oral delivery of nonderivatized testosterone results in negligible blood concentration of testosterone, as substantially complete “digestion” of the hormone occurs in the stomach and liver-putting stress on the liver. In contrast to testosterone, DHEA and progesterone can be taken orally in solid or suspended form, and if enough is taken orally in solid or suspended form, achieve therapeutically effective bloodstream concentrations. However, ingesting either of these nonderivatized hormones in solid or suspended form places significant stress on the liver as the majority of these nonderivatized hormones are digested and not beneficially transferred to the bloodstream. Thus, oral delivery to achieve therapeutically effective bloodstream concentrations is practically non-existent for some nonderivatized hormones, while for others oral delivery results in substantial loss of the hormone—and in either case, undesirable stress is placed on the liver at a minimum, with liver damage being possible. This situation is especially apparent for nonderivatized hormones that are not well-solubilized in water or oil.

Nonderivatized hormone including transdermal creams and gels applied to various locations on the skin, including the underarm, nasal tissues, breast, abdomen, and thigh have been attempted to bypass liver metabolism. However, due to being macroemulsions or pseudo-emulsions, conventional transdermal creams and gels often suffer limitations from relatively slow, poor, and variable rates of absorption, especially over time, and the potential alteration of the hormones during transport through the skin by enzymes in the skin. Furthermore, being applied to the skin, such preparations are often transferred to clothing and other surfaces and can become a danger to other family members.

More recently nonderivatized hormone including solid pellets have been implanted under the skin. The pellets are designed to dissolve in body fluids over time, thus providing a somewhat continuous hormone dose over a 3- to 6-month period. While surgical implantation of the solid pellets is required, injection or daily transdermal application of the hormone is avoided. However, in practice the release of the hormone is often dependent on implant depth, tissue location of the solid pellets, and whether the pellets are undesirably agitated by impact or exercise. In combination, these additional variables, especially undesirable agitation arising from exercise, result in wide variance in the release profile of the hormone—commonly in the form of initial phase of over-dose and a later phase of under-dose. Furthermore, surgical removal of the implant is required if severe over-dosing occurs.

Emulsions are mixtures of two or more liquids that do not solubilize. Thus, the two or more liquids do not form a solution and an identifiable interface exists between the combined liquids. Emulsions may be macroemulsions, pseudo-emulsions, nanoemulsions or microemulsions. Emulsions may be used for parenteral delivery, ocular delivery, transdermal delivery, oral delivery, and the like.

FIG. 1A represents an example nanoemulsion droplet 100 having a single wall of phospholipids (monolayer) forming a hydrophilic exterior 120 and a hydrophobic interior 110. The monolayer wall of the nanoemulsion droplet 100 is formed from a single layer of phospholipids. The outer wall 120 is water-soluble due to the phosphate functionality while the interior 110 is fat-soluble due to the alkyl functionality. FIG. 1B represents multiple of the nanoemulsion droplets 100 in a continuous phase 150.

FIG. 2A represents a microemulsion droplet 200 having a single wall of phospholipids (monolayer) forming a hydrophilic exterior 220 and a hydrophobic interior 210. As with the nanoemulsion droplet 100, the monolayer wall of the microemulsion droplet 200 is formed from a single layer of phospholipids. In relation to the represented nanoemulsion droplet 100, the microemulsion droplet 200 is substantially smaller in diameter-which is often the case for microemulsions. In fact, the diameter of the microemulsion droplet 200 is reduced to where non-polar tails 230 of the monolayer phospholipids are “crushed” into each other, thus forming a more “solid” interior hydrophobic barrier than in the case of the nanoemulsion droplet 100 as represented in FIG. 1. FIG. 2B represents multiple microemulsion droplets 200 in a continuous phase 250. Also represented in the continuous phase 250 are a few individual phospholipid molecules 260 not incorporated into the microemulsion droplets 200.

Conventional transdermal hormone creams are typically “pseudo-emulsions” with solid granules of the nonderivatized hormone not fully solubilized in the droplets of the macroemulsion forming the cream. Others have the nonderivatized hormone dissolved (if oil-soluble) or more commonly dispersed in a carrier oil. In contrast to the larger droplet macro- and pseudo-emulsions, the smaller droplets of nanoemulsions and microemulsions provide the potential to provide better hormone delivery performance than conventionally available from macro- and pseudo-emulsions for transdermal or oral adsorption; however, microemulsions are not readily made for nonderivatized hormones.

While the high-energy forces, temperature, and combinations thereof, used to form nanoemulsions can provide the smaller droplets of a microemulsion, such nanoemulsions are not thermally stable, do not form shelf-stable microemulsions, and are like a macroemulsion in that the components of the nanoemulsion eventually separate into immiscible polar and non-polar liquids. Thus, as represented in FIG. 1 and FIG. 2, nanoemulsion droplets tend to be larger than microemulsion droplets as the nanoemulsion droplets continually expand in diameter after formation until the agglomerating droplets separate from the continuous phase.

Conventionally, macroemulsions, nanoemulsions, and microemulsions have been used for either oil-soluble or water-soluble deliverables, but have had limited success in solubilizing compounds having low solubility in oil and essentially no solubility in water. Deliverables, such as many nonderivatized hormones, have low solubility in oil and essentially no solubility in water, but often have good solubility in alcohol or in mixtures of alcohol and oil. However, if the nonderivatized hormone/alcohol or hormone/alcohol/oil mixture is dispersed along with surfactants into water-based solutions to form an emulsion, the alcohol tends to partition into the water and the nonderivatized hormone solubility enhancement provided by the alcohol, or the alcohol component of the alcohol/oil mixture is lost. This is believed attributable to the alcohol being extremely soluble in the water, in fact especially in relation to the oil if an alcohol/oil mixture is used.

Thus, the nonderivatized hormone loses significant bioavailability in such conventional emulsions, as once solubility in the alcohol or alcohol/oil mixture is lost, the nonderivatized hormone precipitates from the emulsion. In view of this disadvantage, conventionally, there has been little success in the development of oil-in-water (OIW) type microemulsions for nonderivatized hormone delivery, especially in the context of oral or transdermal nonderivatized hormone delivery.

Unlike OIW emulsions (oil droplets in a water continuous phase), conventional water-in-oil emulsions (water droplets in an oil continuous phase—thus, an “invert emulsion”) have been made with nonderivatized hormones. One such example is found in U.S. Pat. Pub. 2009/0069279 (abandoned) to Astruc et al. Astruc describes using nonderivatized dehydroepiandrosterone (DHEA) in an invert emulsion using non-ingestible polar glycolic and hydroglycolic solvents dispersed with silicone-based emulsifiers into an oil medium. The reference recognizes the alcohol-soluble nature of nonderivatized DHEA and the difficulty of incorporating DHEA into an OIW emulsion. However, the WIO systems of Astruc cannot be made for human consumption because of the inedible constituents, thus being limited to transdermal application.

Conventional emulsion delivery systems have traditionally addressed the inability to form true oil-in-water nonderivatized hormone emulsions by first derivatizing the hormone with ester functionality, thus substantially enhancing the oil-solubility of the hormone. The ester groups of the derivatized hormone provide increased oil-solubility to the hormone, thus permitting the esterified hormone to be dissolved in oils for injection or to be carried by conventional oil-in-water emulsion formulations.

A conventional example of hormone derivatization to increase oil solubility is the esterification of the steroidal hormone testosterone. Ester-derivatized testosterone is de-esterified to form bioavailable free testosterone after injection into a living mammal at different rates chiefly due to the release rate of the esterified hormone from the solubilizing excipient oil nodule formed at the injection site. While some variation in the release rate of the esterified hormone from the excipient oil may be attributable to injection technique and tissue variation, a significant factor determining the release rate of the esterified hormone after injection is the nature of the ester group attached to the testosterone.

For example, the propionate ester of testosterone is released from the injected excipient oil nodule much more rapidly than the cypionate ester. Because ester-derivatized testosterone is oil-soluble, in addition to injection with an oil excipient, ester-derivatized testosterone lends itself to conventional oil-in-water emulsion technologies that are used for oil-soluble deliverables. Disadvantages of such conventional methods may include, slowed and sporadic de-esterification of the oil-trapped hormone, stress placed on the liver by the required de-esterification process, the fact that not all hormones can be esterified in high yield, and the added complexity and hormone loss resulting from the esterification reaction.

An issue with conventional delivery systems, including nonderivatized hormone transdermal creams, nonderivatized hormone solid pellet implants, and derivatized hormone injectable oil preparations is that the release profile of the hormone into the bloodstream may not correlate well with the desired hormone dosing profile. Each of these conventional delivery systems is designed to eliminate the need to daily inject the nonderivatized hormone, but in doing so provide a relatively long-term elevated bloodstream concentration of the hormone.

Injections including an excipient oil in combination with the derivatized hormone are designed to prevent having to daily inject the nonderivatized hormone by releasing the derivatized hormone from the oil excipient over time, thus permitting one or two injections per week to maintain a decaying, but somewhat level hormone concentration in the bloodstream. Solid pellet implants are designed to replace weekly or bi-weekly injections with quarterly surgical implants that provide an increasing and then decaying, but somewhat level hormone concentration in the bloodstream during the quarter.

However, research indicates that such elevated blood hormone concentrations over an extended time that slowly increase and/or decrease may not be desired. In fact, such injection of esterified testosterone dissolved in oil or implantation of constant release capsules may generate supraphysiological and/or constantly elevated testosterone concentrations in the blood that fail to provide the desired androgenic effects while increasing the likelihood of undesirable side effects including testicular atrophy and undesirably elevated levels of estradiol.

For example, in “Testosterone in a cyclodextrin-containing formulation: behavioral and physiological effects of episode-like pulses in rats.” (Pharm Res. 1989 July; 6 (7): 641-6) the authors demonstrated in castrated and intact rats that testosterone supplementation should mimic the natural episodic release by the testes to obtain the greatest improvement in androgen-sensitive behavior and physiology. Thus, testosterone supplementation should follow a “pulsed” dosing regimen of multiple high doses that trail off rapidly throughout the day. The study also demonstrated that the testosterone effects were more pronounced when the high pulsed dosages were used periodically rather than when the same total amount of testosterone was equally divided among doses. The study also noted that both spermatogenesis and increased muscle weight were observed in response to the pulsed dosing without substantial enlargement of the prostate.

In combination with other studies, the authors suggest that a testosterone dose that trails off in a slow and protracted manner over the course of a week due to a single injection or the even longer trailing decay provided by the implantation of solid pellets may not be the proper path to optimal testosterone replacement therapy and may contribute to the adverse effects sometimes associated with testosterone replacement therapy.

The dosing regimen used in the study required daily injections of hormone in an inclusion complex. While such dosing could be used for HRT, the required daily injections would be a deterrence to a large percentage of the population in need of HRT. While hormone creams allow for daily use without injection, the slow and variable uptake through the skin does not replicate the pulsed, rapid on-off blood hormone concentrations of the study. Furthermore, in the instance of derivatized hormones, the additional liver toxicity arising from de-esterification would be a further deterrence to using derivatized hormones in such a dosing regimen.

There is an ongoing need for simple and efficient materials and methods for transdermal delivery systems for delivering nonderivatized hormones having poor solubility in oil and essentially no solubility in water to the bloodstream. Conventional emulsion systems have traditionally had disadvantages including poor stability to cold and heat, particularly regarding maintaining the desired average droplet diameter in the emulsion. Maintaining the desired average droplet diameter in the emulsion is important for effective delivery of the nonderivatized hormone to the bloodstream, preventing phase separation of the oil and water emulsion components, and preventing dissociation of the nonderivatized hormone deliverable from the emulsion. In addition to these disadvantages resulting in poor bioavailability of the deliverable, conventional emulsion systems also have the disadvantage of requiring too great a volume of the emulsion in relation to the mass or volume of the deliverable. These disadvantages have been especially true for the transdermal delivery of nonderivatized hormones to mammals, such as humans.

The thickened microemulsions and methods of the present invention overcome at least one of the disadvantages associated with conventional transdermal delivery systems by allowing the convenient, rapid, and efficient transdermal delivery of nonderivatized, directly solubilized hormones to the bloodstream in therapeutically effective amounts. This is an especially significant and previously impractical benefit for transdermal delivery systems if pulsed androgenic activation is the desired dosing regimen.

### SUMMARY

In one aspect, the invention provides a thickened modified oil-in-water microemulsion composition comprising: an alcohol-soluble species; and a thickened modified oil-in-water microemulsion including a modified oil phase and a modified polar continuous phase, where the alcohol-soluble species is solubilized in the modified oil phase, the modified oil phase comprising a phospholipid, a polyethylene glycol derivative, an oil, an alcohol, and an alcohol-lipid phase thickener, and where the modified polar continuous phase includes a continuous phase thickener and water.

In another aspect of the invention, there is a thickened modified oil-in-water microemulsion composition comprising: an alcohol-soluble species; an alcohol-lipid phase thickener, where the alcohol-lipid phase thickener is alcohol-soluble; a continuous phase thickener, where the continuous phase thickener is water-soluble; and a modified oil-in-water microemulsion comprising a modified oil phase and a modified polar continuous phase, where the alcohol-soluble species is solubilized in the modified oil phase, the modified oil phase comprising a phospholipid, a polyethylene glycol derivative, an oil, and an alcohol, and where the modified polar continuous phase comprises water.

In another aspect of the invention, there is a method of making a thickened modified oil-in-water microemulsion composition, the method comprising: combining a phospholipid, a polyethylene glycol derivative, an oil, an alcohol, and an alcohol-lipid phase thickener to form an alcohol-lipid mixture; combining a continuous phase thickener and water to form a modified polar continuous phase; and combining an alcohol-soluble species with the alcohol-lipid mixture and the modified polar continuous phase at atmospheric pressure to form the thickened modified oil-in-water microemulsion.

In another aspect of the invention, there is a method of delivering an alcohol-soluble species to the bloodstream of a human subject, the method comprising: applying a thickened modified oil-in-water microemulsion composition to skin of a human subject, where the composition comprises: an alcohol-soluble species; an alcohol-lipid phase thickener, where the alcohol-lipid phase thickener is alcohol-soluble; a continuous phase thickener, where the continuous phase thickener is water-soluble; and a modified oil-in-water microemulsion comprising a modified oil phase and a modified polar continuous phase, where the alcohol-soluble species is solubilized in the modified oil phase, the modified oil phase comprising a phospholipid, a polyethylene glycol derivative, an oil, and an alcohol, and where the modified polar continuous phase comprises water; and delivering the alcohol-soluble species to the bloodstream of the human subject, where within 90-minutes of the applying the composition to the skin of the human subject, approximately 1 mL of the composition provides the human subject a blood concentration from 2 to 8 ng/ml of the alcohol-soluble species or a metabolite of the alcohol-soluble species over a baseline pre-application bloodstream concentration of the alcohol-soluble species.

In another aspect of the invention, there is a method of treating a female human subject in need of progesterone replacement therapy with a pulsed progesterone dosage regimen, the method comprising: applying a thickened modified oil-in-water microemulsion composition comprising a therapeutically effective amount of progesterone for a treatment duration of at least two weeks, where the applying occurs once daily to the skin, and where the composition comprises: an alcohol-soluble species; an alcohol-lipid phase thickener, where the alcohol-lipid phase thickener is alcohol-soluble; a continuous phase thickener, where the continuous phase thickener is water-soluble; and a modified oil-in-water microemulsion comprising a modified oil phase and a modified polar continuous phase, where the alcohol-soluble species is solubilized in the modified oil phase, the modified oil phase comprising a phospholipid, a polyethylene glycol derivative, an oil, and an alcohol, and where the modified polar continuous phase comprises water; exceeding at least a baseline bloodstream concentration of progesterone of the human subject within 90-minutes of the applying to produce an elevated progesterone blood concentration in the bloodstream of the human subject; reducing the elevated progesterone concentration in the bloodstream of the human subject to approximate the baseline bloodstream concentration in the bloodstream of the human subject within eight hours of the applying; and reducing breast tenderness, bloating, and mood swings in the human subject in relation to the breast tenderness, bloating, and mood swings that would occur when the total amount of progesterone applied over the treatment period is introduced to the skin as a cream lacking the modified oil-in-water microemulsion.

Other compositions, methods, features, and advantages of the invention will be, or will become, apparent to one with skill in the art upon examination of the following figures and detailed description. It is intended that all such additional compositions, methods, features, and advantages be included within this description, be within the scope of the invention, and be protected by the claims that follow.

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1A represents a nanoemulsion droplet having a single wall of phospholipids (monolayer) forming a hydrophilic exterior and a hydrophobic interior.

FIG. 1B represents multiple of the nanoemulsion droplets in a continuous phase.

FIG. 2A represents a microemulsion droplet having a single wall of phospholipids (monolayer) forming a hydrophilic exterior and a hydrophobic interior.

FIG. 2B represents multiple microemulsion droplets represented in a continuous phase.

FIG. 3 represents a method of making a thickened MOIW microemulsion including an alcohol-soluble species deliverable.

FIG. 4 provides the results of a progesterone blood uptake rate and concentration comparison in graphical form for the serum and the cream.

FIG. 5 provides cumulative Area Under the Curve (AUC) values in (ng*min/mL) determined from the progesterone blood uptake rate and concentration comparison in graphical form for the serum and the cream.

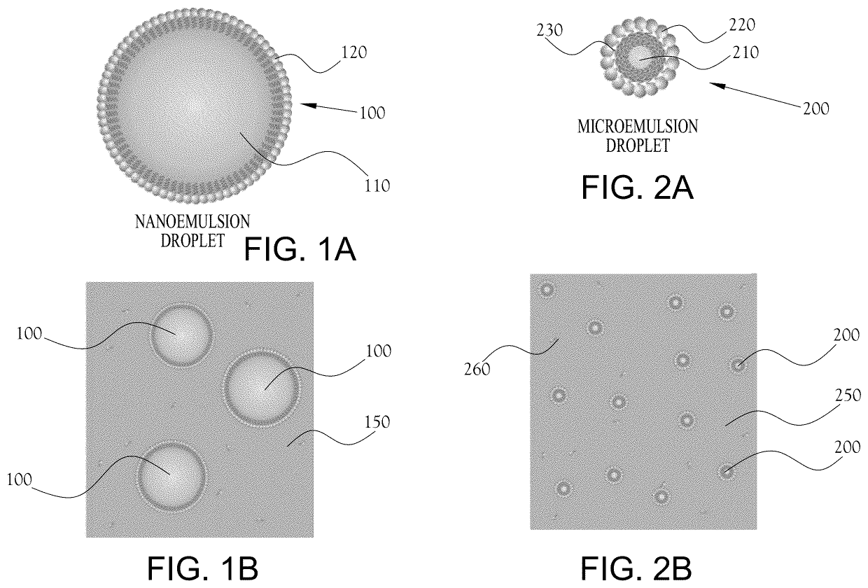

### DETAILED DESCRIPTION

Thickened microemulsions are described where hydrophobic liquid droplets are distributed in a continuous hydrophilic liquid phase. In relation to conventional oil-in-water (OIW) microemulsions, the described thickened microemulsions may be thought of as modified oil-in-water (MOIW) microemulsions, where both the “oil” and “water” phases of the microemulsion are modified. The oil phase droplets of the thickened MOIW microemulsion are modified with alcohol and an alcohol-lipid phase thickener and can solubilize alcohol-soluble species, including nonderivatized hormones. Preferably, the modified oil phase droplets of the thickened MOIW microemulsion directly solubilize nonderivatized hormones. The polar continuous “water” phase of the thickened MOIW microemulsion is modified with a continuous phase thickener. The modified oil phase droplets disperse into the modified polar continuous phase of the thickened MOIW microemulsion.

Unlike the oil phase of a conventional OIW emulsion, the alcohol-lipid phase thickener is believed to reduce the ability of the water of the continuous phase from forming an azeotrope with alcohol. The reduction in the ability of the water of the continuous phase to form an azeotrope with alcohol is believed to reduce the ability of the continuous phase to extract the alcohol from the oil droplets of the alcohol-lipid phase in relation to a water continuous phase. The hydrophobic portion of the monolayer wall formed from the tails of the phospholipid in combination with the polyethylene glycol derivative in the described ratios also is believed to reduce alcohol loss from the oil droplets in relation to conventional OIW emulsions due to the formation of a less porous hydrophobic portion of the monolayer wall.

The retained high alcohol content of the modified oil phase droplets provided by the combination of the alcohol-lipid phase thickener with the hydrophobic monolayer is believed to increase the solubility of the alcohol-soluble species in the modified oil droplets of the thickened MOIW microemulsion in relation to the oil phases of conventional OIW emulsions. This enhanced solubility of the alcohol-soluble species in the modified oil droplets of the thickened MOIW microemulsion is believed to reduce dissociation (e.g. recrystallization, precipitation, and like—thus separation) of the alcohol-soluble species from the oil droplets of the thickened MOIW microemulsion during storage thus making the thickened MOIW microemulsion a shelf-stable microemulsion that preferably is visually clear and more preferably transparent.

The continuous phase thickener is believed to decrease water activity in the modified polar continuous phase via its thickening function, and thus enhance the shelf-stability of the thickened MOIW microemulsion, while reducing the expected alcohol loss from the alcohol-lipid phase.

In the thickened MOIW microemulsion, modified oil phase droplets including the alcohol-soluble species have an average droplet diameter of 1 to 100 nanometers and a preferable average droplet diameter of 5 to 50 nanometers. More preferably, the modified oil phase droplets of the thickened MOIW microemulsion have an average droplet diameter from 7 to 30 nanometers.

The alcohol-soluble species of the thickened MOIW microemulsions is a deliverable that may be delivered trans-mucosal (e.g. intranasal, vaginal, or rectal) or preferably transdermally via the thickened MOIW microemulsion. In addition to directly solubilized nonderivatized hormones, derivatized hormones, such as esterified hormones, may be included in the microemulsion due to their oil-solubility, in the event a greater hormone density in the microemulsion or a modified dosing profile is desired.

The thickened MOIW microemulsion can provide the uptake of the alcohol-soluble species to the bloodstream of a mammal transdermally through the skin. When the alcohol-soluble species is a nonderivatized hormone, such uptake to the bloodstream may be accomplished without the substantial modification and/or transformation of the nonderivatized hormone that has plagued prior, conventional OIW microemulsion transdermal attempts and without substantial stress on the liver.

Preferably, the thickened MOIW microemulsion including the alcohol-soluble species is ingestible and edible. Thus, unlike the WIO microemulsions described in the literature, the described thickened MOIW microemulsions provide therapeutically effective bloodstream concentrations of nonderivatized hormones, including testosterone and progesterone that are ingestible and edible, even though the thickened MOIW microemulsions are not preferred for oral delivery.

The ability of the thickened MOIW microemulsion to deliver alcohol-soluble species rapidly, efficiently, and without substantial modification and/or transformation provides for pulsed dosing regimens not practical with conventional transdermal delivery systems having relatively slow, poor, inconsistent, and inefficient bloodstream delivery. For example, the benefits of a pulsed dosing regimen for testosterone are suggested from prior animal studies, where daily injected doses of testosterone were found to mimic the natural episodic release of testosterone from the testes and provided improvements in androgen-sensitive behavior and physiology. In contrast, introducing the same total amount of testosterone supplied by the multiple, daily injections as a single dose that is slowly released over an extended time resulted in unnatural, constantly elevated testosterone blood concentrations, which was suggested to be a cause of undesirable side effects associated with testosterone HRT.

A pulsed testosterone dosing regimen made possible by the thickened MOIW microemulsion would include a daily, morning, transdermal dose of testosterone. As testosterone is rapidly metabolized from the blood with about 90% metabolized within the first hour of introduction and the remainder metabolized within three hours, elevated blood testosterone levels would not exist but for a few hours each morning. The very short, elevated period in relation to the very long unnatural dosing regimen provided by conventional preparations should substantially reduce testicular atrophy and other undesirable side effects attributable to continuously elevated blood testosterone levels. While such a pulsed testosterone dosing regimen can be implemented through daily injection, such a regimen is made possible by the thickened MOIW microemulsion without injection.

The thickened MOIW microemulsion allows for pulsed dosing regimens of nonderivatized hormones other than testosterone. For example, a pulsed progesterone dosing regimen made possible by the thickened MOIW microemulsion would include a daily, morning or evening, transdermal dose of progesterone. Elevated blood progesterone levels would not exist but for approximately six to eight hours during the day or night. Thus, the unwanted side effects from extended, elevated blood concentrations associated with conventional daily progesterone use, including breast tenderness, bloating, and mood swings may be reduced, while the benefits of daily use, including the elimination of cycle bleeding, are maintained. While such a pulsed progesterone dosing regimen could be implemented through daily injection, such a regimen is made possible by the thickened MOIW microemulsion without injection. Other nonderivatized hormones may be similarly pulse dosed with the thickened MOIW microemulsion.

The thickened MOIW microemulsion preferably includes a ratio of phospholipid, to oil, to polyethylene glycol derivative, to alcohol, to alcohol-lipid phase thickener, to continuous phase thickener, and to water of 1:1:0.6-3.3:1.8:1.8:0.02-2.5:7 by weight, with deviations up to 20% by weight being included, and with deviations up to 10% by weight being more preferred, thus 1:1:0.6-3.3:1.8:1.8:0.02-2.5:7±20% by weight or 1:1:0.6-3.3:1.8:1.8:0.02-2.5:7±10% preferred by weight.

The alcohol-soluble species is preferably included in the thickened MOIW microemulsion at a ratio of oil to alcohol-soluble species of 1:0.02 to 1:0.5 by weight, with a ratio of oil to alcohol-soluble species of 1:0.02 to 1:0.3 by weight being preferred with deviations up to 10% by weight being included, and with deviations up to 5% by weight being more preferred, thus 1:0.02 to 1:0.3±10% by weight or 1:0.02 to 1:0.3±5% preferred by weight.

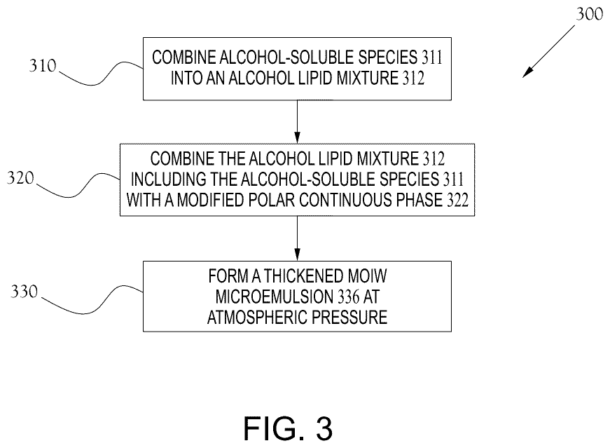

FIG. 3 represents a method 300 of making a thickened MOIW microemulsion 336 including an alcohol-soluble species 311 deliverable. In 310, the alcohol-soluble species 311 is combined into an alcohol-lipid mixture 312 including a polyethylene glycol derivative, a phospholipid, an oil, an alcohol, and an alcohol-lipid phase thickener. In 320, the alcohol-lipid mixture 312 including the alcohol-soluble species 311 is combined with a modified polar continuous phase 322 including a continuous phase thickener and water. The alcohol-lipid mixture 312 including the alcohol-soluble species 311 may be considered a modified oil phase dispersed in the modified polar continuous phase 322, which may be thought of as a modified water phase.

Alternatively, the alcohol-lipid phase thickener may be wholly or partially omitted from the alcohol-lipid mixture 312 and instead added to the modified polar continuous phase 322—if the alcohol-lipid phase thickener has sufficient water-solubility in addition to the desired alcohol-solubility. In this instance the alcohol-lipid mixture 312 is added to the modified polar continuous phase 322 including the alcohol-lipid phase thickener in addition to the continuous phase thickener and water.

In 330, the thickened MOIW microemulsion 336 including the alcohol-soluble species 311 is formed by mixing at atmospheric pressure. Unlike in nanoemulsions, the thickened MOIW microemulsion 336 may be formed at atmospheric pressure without needing high-energy to form. Although the thickened MOIW microemulsion 336 could be formed using high-energy forces as used in forming nanoemulsions, the result eventually will be the thickened MOIW microemulsion 336, as unlike in a nanoemulsion that begins the dissociation process after formation, the thickened MOIW microemulsion 336 is thermally stable at room temperature and pressure after formation. Thus, formation of the thickened MOIW microemulsion 336 dispenses with the undesirable use of high-energy forces during formation and is shelf-stable, preferably visually clear, and more preferably transparent after formation.

While the method 300 represents the alcohol-soluble species 311 first being combined with the alcohol-lipid mixture 312, the alcohol-lipid mixture 312 and the modified polar continuous phase 322 may first be combined and the alcohol-soluble species 311 then added to form the thickened MOIW microemulsion 336 (not shown). This step rearrangement is possible as the modified oil and modified polar continuous phases will “self-assemble” droplets including the alcohol-soluble species to form the thickened MOIW microemulsion 336 at atmospheric pressure.

In addition to the alcohol-soluble species 311, the thickened MOIW microemulsion 336 may include additional deliverables that are soluble in water or oil. However, in addition to transdermally delivering water and/or oil soluble deliverables to the bloodstream, the thickened MOIW microemulsion 336 has the unexpected ability to transdermally deliver therapeutically effective concentrations of the alcohol-soluble species to the bloodstream of a subject more rapidly and with significantly greater delivery efficiency than conventional transdermal creams.

The alcohol-soluble species 311 includes nonderivatized hormones, polyphenols, plant sterols, and amines singularly or in combination. The alcohol-soluble species is solubilized in the droplets of the thickened MOIW microemulsion 336, thus in the alcohol-lipid mixture 312. Preferably, the alcohol-soluble species 311 constitutes from 0.2% to 5% of the thickened MOIW microemulsion 336 by weight. However, to provide a visually clear thickened MOIW microemulsion 336 with the widest range of alcohol-soluble species, weight percentages of the alcohol-soluble species 311 from 0.2% to 3% are preferred, with weight percentages from 0.25% to 3% being more preferred. For nonderivatized hormones, weight percentages in the thickened MOIW microemulsion 336 from 0.2% to 1.8% are readily achieved, with a weight percent from 0.25% to 1.5% being readily achieved for nonderivatized testosterone and for nonderivatized progesterone.

Preferable alcohol-soluble nonderivatized hormones include testosterone, dehydroepiandrosterone (3-beta-hydroxyandrosteron-5-en-17-one) (DHEA), dihydrotestosterone (DHT), 7-keto DHEA, pregnenolone, androstenedione (AD), androstenediol, progesterone, estradiol, estrone, estriol, cortisol, and hydrocortisone. More preferred nonderivatized hormones are testosterone, DHEA, and progesterone. At present, the most preferred nonderivatized hormones are testosterone and progesterone in micronized powder form. Preferable alcohol-soluble polyphenols include chrysin, hesperetin, apigenin, and epigallocatechin gallate (EGCG). Preferable alcohol-soluble plant sterols include Tribulus terrestris and yohimbe, while preferable alcohol-soluble amines include diindolylmethane (DIM).

The alcohol lipid mixture 312 may include an oil-soluble deliverable specie or species that are more soluble in oil than the alcohol-soluble species 311. Such oil-soluble deliverables are solubilized in the modified oil phase droplets of the microemulsion, thus in the alcohol-lipid mixture 312 with the alcohol-soluble species 311.

Oil-soluble deliverable species include derivatized hormones, cannabis extracts, terpenes, and oil-soluble vitamins. Preferable derivatized hormones include testosterone-propionate, testosterone-cypionate, testosterone-enanthate, and testosterone-phenylpropionate. More preferred derivatized hormones are testosterone-propionate and testosterone-cypionate. At present, the most preferred derivatized hormone is testosterone-cypionate.

Preferable cannabis extracts include cannabidiol (CBD), tetrahydrocannabinol (THC), and other cannabinoids including cannabinol (CBN), cannabigerol (CBG), tetrahydrocannabivarin (THCV), cannabidivarin (CBDV), and cannabichromene (CBC). Preferable terpenes include monoterpenes (incorporate two isoprene units and have the molecular formula C₁₀H₁₆), monoterpenoids, diterpenes (incorporate four isoprene units and often have the molecular formula C₂₀H₃₂), and diterpenoids. Preferable terpenes include limonene, pinene, linalool, beta-caryophyllene, retinol, phytol, myrcene, humulene, ocimene, terpinolene, geraniol, and geranylgeraniol.

Preferable oil-soluble vitamins include Vitamin A, Vitamin D, Vitamin E, Vitamins K1, and Vitamin K2. Oil-soluble carotenoids, such as lutein, zeaxanthin, lycopene, and beta carotene also may be included, but are not preferred due to their potential to stain the skin.

The modified polar continuous phase 322 may include a water-soluble deliverable specie or species that is more soluble in water than the alcohol-soluble species 311. Such water-soluble deliverables are solubilized in the modified polar continuous phase 322 of the thickened MOIW microemulsion 336, thus, in the carrier liquid of the thickened MOIW microemulsion 336. Preferred water-soluble deliverables include Vitamin C, Vitamin B5, Nicotinamide MonoNucleotide (NMN), Nicotinamide Adenine Dinucleotide (NAD), Glutathione, nitric oxide, L-citrulline, L-carnosine, aloe, and beta-Hydroxy beta-Methyl Butyric acid (HMB).

The phospholipid and the polyethylene glycol derivative in combination form the boundary between the modified polar continuous phase 322 and the interior of the modified oil phase droplets of the thickened MOIW microemulsion 336. To maintain the desired alcohol concentration within the droplets, thus reducing the likelihood of losing the alcohol to the modified polar continuous phase and the associated dissociation of the alcohol-soluble species from the droplets, the phospholipid, polyethylene glycol derivative, and the ratio between the two are important, as previously discussed.

The phospholipid of the alcohol-lipid mixture 312 is a glycerophospholipid preferably isolated from lecithin. As the phospholipid is preferably a lecithin isolate, the named isolates preferably include 80% (w/w) of the specified phospholipid with the remaining constituents being one or more additional phospholipids isolated from the lecithin or other lecithin isolates.

Preferred phospholipid lecithin isolates include phosphatidylcholine (PC), phosphatidylethanolamine (PE), phosphatidylinositol (PI), ceramide phosphoryl ethanolamine (Cer-PE), ceramide phosphoryl choline (SPH), and combinations thereof, with PC, PE, and combinations thereof being more preferred. However, all phospholipid lecithin isolates are unexpectedly not interchangeable in forming visually clear, shelf-stable thickened MOIW microemulsions, as the phosphatidylserine (PS) and phosphatic acid (PA) isolates are not useful when both visually clear and shelf-stable thickened MOIW microemulsions are desired.

When the alcohol-soluble species 311 is nonderivatized testosterone, the phospholipid is preferably PC. When the alcohol-soluble species 311 is nonderivatized progesterone, the phospholipid is more preferably PC90, thus where the lecithin isolate is approximately 90% (w/w) PC.

The phospholipid may be present in the thickened MOIW microemulsion 336 from 3% to 10% on a weight basis. Preferably, the phospholipid constitutes from 4% to 8% of the thickened MOIW microemulsion 336 on a weight basis. When the alcohol-soluble species is nonderivatized testosterone or nonderivatized progesterone, the phospholipid constitutes from 6% to 8% of the thickened MOIW microemulsion 336 on a weight basis.

The polyethylene glycol derivative of the alcohol-lipid mixture 312 may be a polyethylene glycol modified vitamin E, such as tocopheryl polyethylene glycol succinate 1000 (TPGS), polysorbate 40, polysorbate 60, or polysorbate 80. Preferably, the polyethylene glycol derivate is TPGS, polysorbate 60, or polysorbate 80. More preferably, the polyethylene glycol derivative is TPGS or polysorbate 80. When the alcohol-soluble species is nonderivatized testosterone or nonderivatized progesterone, the preferred polyethylene glycol derivative is TPGS.

The polyethylene glycol derivative may be present in the thickened MOIW microemulsion 336 from 5% to 14% on a weight basis. Preferably, the polyethylene glycol derivative constitutes from 6% to 12% of the thickened MOIW microemulsion 336 on a weight basis. When the alcohol-soluble species is nonderivatized testosterone or nonderivatized progesterone, the polyethylene glycol derivative constitutes from 6% to 10% of the thickened MOIW microemulsion 336 on a weight basis.

When used in conjunction with the phospholipid, TPGS resulted in visually clear, shelf-stable microemulsions at phospholipid to TPGS ratios of approximately 1:0.4 to 1:5 by weight, with preferred shelf-stable, transparent thickened MOIW microemulsions being formed at ratios of 1:0.8 to 1:1.5 by weight. TPGS and polysorbate 80 are the presently preferred polyethylene glycol derivatives as in combination with the phospholipid they provide the desired visually clear, shelf-stable microemulsions over the widest alcohol-soluble species concentration range.

The alcohol-lipid mixture 312 preferably includes at least one oil held within the phospholipid/polyethylene glycol derivative monolayer. The oil may be an MCT oil, a citrus oil, and combinations thereof. Preferable MCT oils include caproic acid (hexanoic acid), caprylic acid (octanoic acid), capric acid (decanoic acid), lauric acid (dodecanoic acid), and combinations thereof. More preferred MCT oils include caprylic acid, capric acid, and combinations thereof. Preferred citrus oils include orange oil, lemon oil, and combinations thereof. When the alcohol-soluble species is nonderivatized testosterone or nonderivatized progesterone, the oil is preferably a combination of caprylic and capric acids.

The oil may be present in the thickened MOIW microemulsion 336 from 5% to 15% on a weight basis. Preferably, the oil constitutes from 7% to 13% of the thickened MOIW microemulsion 336 on a weight basis. When the alcohol-soluble species is nonderivatized testosterone or nonderivatized progesterone, the oil constitutes from 6% to 10% of the thickened MOIW microemulsion 336 on a weight basis.

The thickened MOIW microemulsion 336 includes at least one alcohol-lipid phase thickener. The alcohol-lipid phase thickener is alcohol-soluble, but preferably has significant water-solubility in addition to the alcohol-solubility. An advantage of the alcohol-lipid phase thickener being alcohol- and significantly water-soluble is that it can be added to the alcohol-lipid mixture 312 and/or the modified polar continuous phase 322 during formation of the thickened MOIW microemulsion 336.

Preferable alcohol-lipid phase thickeners are glycols and alkyl benzoates. Preferable glycols include butylene glycol (1,3-butanediol), propylene glycol, hexylene glycol, ethoxydiglycol, and di-propylene glycol, with butylene glycol being more preferred at present. Preferable alkyl benzoates include C12-15 alkyl benzoate.

The alcohol-lipid phase thickener may be present in the thickened MOIW microemulsion 336 from 8% to 16% on a weight basis. Preferably, the alcohol-lipid phase thickener constitutes from 10% to 14% of the thickened MOIW microemulsion 336 on a weight basis.

The ratio of the alcohol-lipid phase thickener to the oil is from 1:0.3 to 1:1.5 by weight, preferably 1:0.6 by weight with deviations up to 20% by weight being included, and with deviations up to 10% by weight being more preferred, thus 1:0.6±20% by weight or 1:0.6±10% preferred by weight.

Other ratios of alcohol-lipid phase thickener to oil result in a microemulsion that loses transparency, visual clarity, and/or is no longer shelf-stable. It was unexpected that the described alcohol-lipid phase thickener to oil ratios were able to reduce the loss of alcohol from the alcohol-lipid phase to the point where a shelf-stable thickened MOIW microemulsion could be formed that was shelf-stable, preferably visually clear, and more preferably transparent.

The thickened MOIW microemulsion 336 includes at least one alcohol. The preferable alcohol is food grade as the thickened MOIW microemulsion 336 is preferably edible, though intended for application to the skin. Preferably, the alcohol is ethanol, with USP food grade 190 proof (95% ethanol, 5% water) ethanol being more preferred. Alcohol water contents greater than 10% by weight are less preferred, as then the additional water should be considered in relation to the total water content of the thickened MOIW microemulsion 336 to prevent dissociation of the alcohol-soluble species from the modified oil phase droplets.

The alcohol may be present in the thickened MOIW microemulsion 336 from 5% to 25% on a weight basis. Preferably, the alcohol constitutes from 10% to 23% of the thickened MOIW microemulsion 336 on a weight basis. When the alcohol-soluble species is nonderivatized testosterone or nonderivatized progesterone, the alcohol constitutes from 11% to 14% of the thickened MOIW microemulsion 336 on a weight basis.

The modified oil phase droplets of the thickened MOIW microemulsion 336 may be considered to have a high alcohol content, thus having an oil to alcohol weight ratio from 1:0.8 to 1:3, preferably from 1:1.2 to 1:2 by weight.

The modified polar continuous phase 322 includes a continuous phase thickener and water. The continuous phase thickener is water-soluble. Preferable continuous phase thickeners include salts of hyaluronic acid, such as the sodium salt of hyaluronic acid and the potassium salt of hyaluronic acid, xanthin gum, and acacia gum. Hyaluronic acid is more preferred at present as unlike xanthin and acacia gums, it provides a smooth texture when applied to the skin. The sodium salt of hyaluronic acid is most preferred at present.

Unexpectedly, while being water-soluble, carbomer, hydroxyethyl cellulose, and guar gum-based thickeners were unable to assist in preventing dissociation of the alcohol-soluble species from the alcohol-lipid phase, likely attributable to alcohol loss from the alcohol-lipid phase, thus resulting in dissociated emulsions that were not transparent, visually clear, or shelf-stable.

The continuous phase thickener may be present in the thickened MOIW microemulsion 336 from 0.15% to 5% on a weight basis. Preferably, the continuous phase thickener constitutes from 0.25% to 4% of the thickened MOIW microemulsion 336 on a weight basis. When the continuous phase thickener is a salt of hyaluronic acid, the continuous phase thickener may be present in the thickened MOIW microemulsion 336 from 0.15% to 1.5%, preferably from 0.25% to 0.75%, on a weight basis. When the alcohol-soluble species is nonderivatized progesterone and the continuous phase thickener is the sodium salt of hyaluronic acid, the continuous phase thickener constitutes from 0.28% to 0.38% of the thickened MOIW microemulsion 336 on a weight basis.

The ratio of the continuous phase thickener to the alcohol-lipid phase thickener is from 1:2.5 to 1:50 by weight, preferably 1:5 to 1:40 by weight. When the continuous phase thickener is the sodium salt of hyaluronic acid, the ratio of the continuous phase thickener to the alcohol-lipid phase thickener is from 1:30 to 1:50 by weight, preferably 1:40 by weight with deviations up to 20% by weight being included, and with deviations up to 10% by weight being more preferred, thus 1:40±20% by weight or 1:40±10% preferred by weight

The water of the modified polar continuous phase 332 is present in the thickened MOIW microemulsion 336 from 47% to 55% on a weight basis. Preferably, water is present from 48% to 54% on a weight basis in the thickened MOIW microemulsions 336. More preferably, water may be present in the thickened MOIW microemulsion 336 from 49% to 52% on a weight basis.

The thickened MOIW microemulsion 336 may optionally include other ingredients or “adjuvants” that are chemically compatible with the alcohol-soluble species and do not substantially interfere with the separation between the thickened, modified oil and water phases of the thickened MOIW microemulsion 336. Such adjuvants may include preservatives, antioxidants, electrolytes, perfumes, fillers, and pigments. Other adjuvants may be used in the thickened MOIW microemulsion 336.

In combination, the hyaluronic acid and butylene glycol thickeners added to the modified water and modified oil phases of the emulsion provide the thickened MOIW microemulsion 336 or “serum” a viscosity from 300 centipoise (cp) to 500 cp, preferably from 350 cp to 550 cp, and more preferably from 360 cp to 460 cp. In a most preferred aspect, the viscosity of the thickened MOIW microemulsion 336 is from 390 cp to 430 cp.

In combination, the alcohol-lipid and continuous phase thickeners increase the viscosity of the thickened MOIW microemulsion 336 in relation to a microemulsion without the thickeners but having a significant sugar or sugar alcohol content in the continuous phase from approximately 250 cp to 270 cp (a microemulsion having significant sugar or sugar alcohol content in the continuous phase) to the 360 cp to 460 cp range (thickened MOIW microemulsion 336). Thus, an approximately 45% increase in viscosity is observed for the thickened MOIW microemulsion 336. However, the thickened MOIW microemulsion 336 as thickened by the alcohol-lipid and continuous phase thickeners is not as viscous as conventional creams and lotions, which have viscosities greater than 2000 cp. Thus, the thickened MOIW microemulsion 336 may be referred to as a serum instead of a less-viscous liquid or more-viscous cream, lotion, or gel.

The relatively low viscosity of the thickened MOIW microemulsion 336 in relation to conventional hormone creams in combination with the use of the selected thickeners provide the thickened MOIW microemulsion 336 a short skin residence time of 3 to 8 minutes, preferably from 3 to 4 minutes, after which time the ability to “feel” or transfer the serum to other surfaces is significantly reduced.

The short skin residency time of the thickened MOIW microemulsion 336 reduces the likelihood that the subject will inadvertently transfer the alcohol-soluble species to unwanted areas as is a common disadvantage attributed to conventional testosterone and progesterone creams. The fact that the thickened MOIW microemulsion 336 provides rapid and efficient bloodstream delivery to the alcohol-soluble species is unexpected in relation to conventional macroemulsion and pseudo-emulsion-based creams and gels.

The following examples are provided to illustrate one or more preferred embodiments of the invention. Numerous variations can be made to the following examples that lie within the scope of the invention.

### EXAMPLES

### Example 1: Constituents of a Thickened MOIW Microemulsion Including the Nonderivatized Hormone Progesterone

A thickened MOIW microemulsion was prepared having a 1 mL total volume. The thickened MOIW microemulsion included approximately 8 mg of the nonderivatized hormone progesterone in micronized form. The thickened MOIW microemulsion also included from 50 mg to 100 mg of PC, from 80 mg to 160 mg of ethanol, from 2 mg to 5 mg of hyaluronic acid, from 100 mg to 150 mg of butylene glycol, and from 50 mg to 100 mg of medium chain triglycerides. TPGS was included to provide the desired physical structures in the thickened MOIW microemulsion. In addition to these ingredients, the thickened MOIW microemulsion included enough water to provide a total emulsion volume of 1 mL.

### Example 2: A Method of Making a Thickened MOIW Microemulsion Including the Nonderivatized Hormone Progesterone

Approximately 8 mg of nonderivatized progesterone was combined in MCT oil and then combined with TPGS, PC, ethanol, and butylene glycol to form a thickened modified alcohol-lipid phase. Hyaluronic acid was combined with water to form a thickened continuous phase. The thickened phases were then combined and mixed to form a thickened MOIW microemulsion including the nonderivatized hormone progesterone having a total volume of 1 mL.

### Example 3: Comparative Blood Uptake Rates and Concentrations for Progesterone

A thickened MOIW microemulsion (serum) in accord with Examples 1 and 2 and a commercially available conventional progesterone cream (cream) were the alcohol-soluble species delivery systems used in the comparison. The uptake data for the cream was taken from Herman, “Over-the-Counter Progesterone Cream Produces Significant Drug Exposure Compared to a Food and Drug Administration-Approved Oral Progesterone Product”, J. Clin. Pharmacol. 2005; 45:614-619.

While the nonderivatized hormone progesterone was used as the alcohol-soluble species for delivery by the serum in the Examples, the blood uptake data provided by the serum for the nonderivatized progesterone is believed applicable for alcohol-soluble species other than nonderivatized progesterone, such as for the nonderivatized hormones testosterone and DHEA.

The conventional commercially available cream was stated on the label to include 450 mg of USP progesterone per ounce, thus 900 mg per two-ounce tube, with each approximate 1.25 mL including 20 mg of the USP progesterone. According to the label, the cream also included Water (Purified), Tocopheryl Acetate (Vitamin E), Aloe barbadensis (Aloe Vera) Leaf Juice, Carthamus tinctorius (Hybrid Safflower) Seed Oil, Panthenol, Glycerin, Prunus mmygdalus dulcis (Sweet Almond) Oil, Glyceryl Stearate, Cetyl Alcohol, Sodium Behenoyl Lactylate, Stearic Acid, Phenoxyethanol, Caprylyl Glycol, Sorbic Acid, Carbomer, and Potassium Hydroxide. The cream was a conventional OIW type macroemulsion.

Human subjects applied the cream in an amount to provide 40 mg of progesterone (2.5 mL) twice daily to the skin, thus 80 mg total in a 24-hour period. The skin location was rotated between the inner arm, breast, abdomen, and thigh. The area of application was not occluded or washed after application of the cream. While the cream was used twice daily in the Herman reference, the blood concentrations used for comparison with the serum throughout the Examples were obtained after a single skin application on the 12th day of application, thus being comparable with the single application of the serum.

The serum was applied once in an amount to provide 20 mg of progesterone (2.5 mL) to the skin. The skin location was the inner arm, and the area of application was not occluded or washed after application of the serum. Thus, per unit volume for the application prior to the blood draws, the cream included twice the amount of progesterone as the serum.

Blood samples were collected from the subjects before application of the delivery systems to the skin and at varying time intervals between approximately 20- and 360- or 720-minutes after application. The blood samples collected for the serum were analyzed for the concentration of progesterone using LCMS.

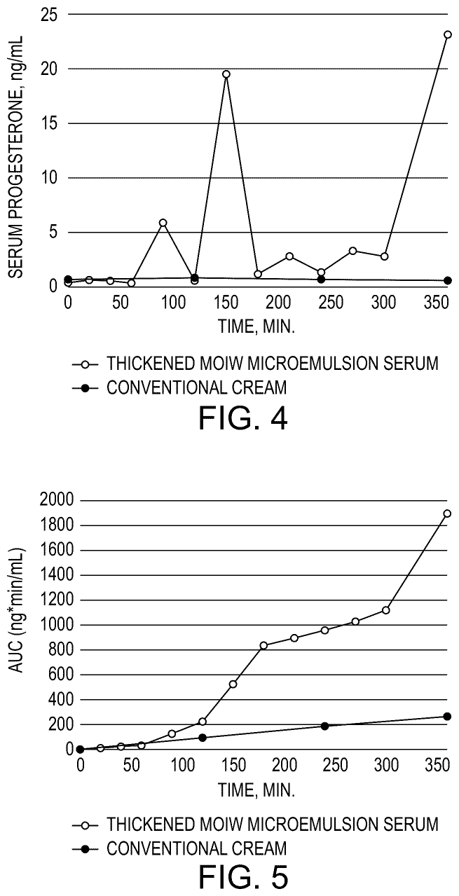

FIG. 4 provides the results of a progesterone blood uptake rate and concentration comparison in graphical form for the serum and the cream. The time after application of the delivery systems by the human subjects when the blood sample was collected is represented on the X-axis, while the average nanograms (ng) of progesterone per milliliter (mL) determined for the blood samples is represented on the Y-axis.

The superiority of the serum at rapidly delivering progesterone to the bloodstream in relation to the cream is apparent from a blood uptake perspective, where an initial spike to approximately 6 ng/mL was observed approximately 90-minutes after application and a second approximately 150 ng/mL spike was observed approximately 150-minutes after application. In contrast, the cream was unable to achieve blood uptake much above the 0.7 ng/mL baseline progesterone bloodstream concentration measured prior to application during the approximately 360-minutes post-application.

For example, at 150-minutes after application, the serum provided a progesterone bloodstream concentration of 19.51 ng/mL while the cream provided a progesterone bloodstream concentration of 0.85 ng/mL 120-minutes after application. Thus, at 150-minutes post-application, the serum delivered nearly 23 times as much progesterone to the bloodstream of the subject as the cream. The ability of the serum to rapidly deliver progesterone at a significantly enhanced rate to the bloodstream in relation to the cream was established.

The serum can provide a human subject with a progesterone blood concentration of at least 2 to 8 ng/mL, preferably at least 4 to 8 ng/mL, within 90-minutes of applying the serum to the skin. The serum can provide a human subject with a progesterone blood concentration of at least 15 to 22 ng/ml, preferably at least 16 to 22 ng/mL, within 180-minutes of applying the serum to the skin.

### Example 4: Total Exposure of Progesterone

FIG. 5 provides cumulative Area Under the Curve (AUC) values in (ng*min/mL) determined from the progesterone blood uptake rate and concentration comparison in graphical form for the serum and the cream. The AUC values provide a measure of the cumulative amount of progesterone in the bloodstream, thus total exposure across a time duration.

Table 1 below shows the blood concentration and AUC values in relation to time for the serum.

TABLE 1

| Time | Serum, | Serum |
|---|---|---|
| Minutes | (ng/mL) | AUC |
| 0 | 0.4 | 0 |
| 20 | 0.64 | 10.4 |
| 40 | 0.57 | 22.5 |
| 60 | 0.36 | 31.8 |
| 90 | 5.9 | 125.7 |
| 120 | 0.59 | 223.05 |
| 150 | 19.51 | 524.55 |
| 180 | 1.19 | 835.05 |
| 210 | 2.82 | 895.2 |
| 240 | 1.35 | 957.75 |
| 270 | 3.32 | 1027.8 |
| 300 | 2.81 | 1119.75 |
| 360 | 23.12 | 1897.65 |

Table 2 below shows the blood concentration and AUC values in relation to time for the cream as estimated from the data provided in the Herman publication.

TABLE 2

| Time | Serum, | Serum |
|---|---|---|
| Minutes | (ng/mL) | AUC |
| 0 | 0.7 | 0 |
| 120 | 0.85 | 93 |
| 240 | 0.7 | 186 |
| 360 | 0.6 | 264 |
| 480 | 0.55 | 333 |
| 600 | 0.8 | 414 |
| 720 | 0.5 | 492 |

At 120-minutes where direct comparison could be made, the cumulative delivery of the cream was 93, while cumulative delivery of the serum was 223, an approximate 136% increase (223−93/93*100%) for the serum. Thus, two hours after transdermal application the serum delivered more than twice as much nonderivatized progesterone to the bloodstream in comparison to the cream. At the 360-minute duration after transdermal application, cumulative delivery of the cream was 264, while cumulative delivery of the serum was 1898, an approximate 619% increase (1898−264/264*100%) for the serum. Thus, six hours after transdermal application the serum delivered more than six times as much nonderivatized progesterone to the bloodstream in comparison to the cream.

Interestingly, even after 720-minutes (twelve hours) the cumulative delivery of the cream was approximately a quarter of that delivered by the serum in six hours, one-half the time. These numbers establish that the serum provided a bloodstream cumulative exposure to the human subject after approximately 2.5 hours that the cream could not quite match after 12 hours. In relation to the cream, the ability of the serum to deliver therapeutically effective bloodstream concentrations of progesterone to the bloodstream was established.

The relatively rapid timeframe in which the serum transdermally delivers a therapeutically effective bloodstream concentration of progesterone permits a pulsed dosing regimen to be used, something that is not possible with the delivery profile of the cream. Thus, relatively small, repeated applications of the serum could be used to replace a larger application, a dosing regimen not possible with the cream due to the low and slow bloodstream delivery.

### Example 5: Progesterone Bloodstream Delivery Efficiency

The cream included 40 mg of nonderivatized progesterone while the serum included 20 mg, thus the serum included half as much nonderivatized progesterone per unit volume in relation to the cream. When the amount of nonderivatized progesterone applied to the skin as opposed to the volume of the cream or serum are considered the bloodstream delivery efficiency of the cream is very poor.

From a bloodstream delivery efficiency perspective, the serum delivered approximately four times as much of the applied nonderivatized progesterone to the bloodstream than the cream after two hours and approximately twelve times as much after six hours. When the twelve-hour cumulative delivery is considered, the cream delivered about one-eighth as much of the transdermally applied nonderivatized progesterone to the bloodstream in comparison to the serum.

For the cream to approximate the cumulative progesterone delivery of the serum over a 12-hour period, one would seemingly have to apply approximately 20 mL of the cream including 320 mg of nonderivatized progesterone to match what 2.5 mL of the serum including only 20 mg of nonderivatized progesterone could deliver. Thus, in addition to the cream requiring daily application in a quantity associated with a whole-body moisturizer, 300 mg of nonderivatized progesterone is wasted every day.

The serum provides a significant production cost advantage over the cream as in relation to the serum, approximately 94% of the progesterone included in the cream is “wasted” as it fails to provide additional relevant bloodstream concentration. In relation to the cream, the ability of the serum to efficiently deliver transdermally applied nonderivatized progesterone to the bloodstream of a subject was established.

### Example 6: Relative Progesterone Bioavailability

Relative nonderivatized progesterone bioavailability between the cream and serum delivery systems was determined at different times after transdermal application by multiplying the AUC up to the selected time point by the amount of nonderivatized progesterone in the selected delivery system and dividing by a control value likewise calculated, thus (AUC1*carrier system1)/(AUCcontrol*carrier system control) at the selected time-thus calculating AUC as a cumulative amount of progesterone in the bloodstream. As this calculation divides the total amount of progesterone delivered by the numerator delivery system by the total amount of progesterone delivered by the denominator delivery system at a selected time, the resulting values relate to how many times more progesterone was delivered by the numerator delivery system in relation to the denominator delivery system at the selected time. Thus, the fact that the cream included twice as much progesterone as the serum per unit volume is normalized.

The results from the AUC calculations are provided below in Table 3 for the serum as the numerator and the cream as the denominator with estimation due to the variability in the withdrawal time of the blood samples.

TABLE 3

| | Bio- |
|---|---|
| | Availability, |
| Time Minutes | F |
| 120 | 4.8 |
| 240 | 10.3 |
| 360 | 14.4 |

At 120-minutes after application to the skin, the serum delivered nearly five times as much progesterone on a cumulative basis to the bloodstream of the subject as did the cream from the same 20 mg of applied nonderivatized progesterone. After four hours, the serum delivered 10 times as much progesterone on a cumulative basis to the bloodstream, while after six hours the serum delivered 14 times as much cumulatively. These calculated values confirm the ability of the serum to deliver therapeutically effective bloodstream concentrations of progesterone rapidly and efficiently to the subjects in relation to the cream.

The serum was able to deliver from 3 to 6 times more nonderivatized hormone to the bloodstream of a subject on a cumulative basis after two hours than the cream. The serum also was able to deliver from 12 to 17 times more nonderivatized hormone to the bloodstream of a subject on a cumulative basis after six hours than the cream.

### Example 7: Pulsed Dosing for Transdermal Application of Nonderivatized Progesterone to Post-Menopausal Female Subjects

Post-menopausal female human subjects in need of progesterone HRT therapy applied approximately 1 mL of the serum including approximately 8 mg of nonderivatized progesterone daily to the inner arm, in the morning or night. Blood progesterone exceeded baseline bloodstream concentration within 90-minutes of application, and at least doubled baseline bloodstream concentration within 90-minutes of application. Unlike a conventional cream, the delivery profile of the serum allowed bloodstream progesterone concentration to return to an approximate baseline bloodstream concentration within eight hours of application of the serum, and preferably within six hours of application.

The pulsed dosing regimen was used to replace or supplement the normal cyclic progesterone levels on the 14^{th} to 28^{th} days of the menstruation cycle of the post-menopausal subjects. The pulsed dosing regimen also can be used to supplement the normal cyclic progesterone levels on days 1 through 14 of the menstruation cycle of pre-menopausal subjects to reduce cycle bleeding.

The serum pulsed dosing regimen significantly reduced breast tenderness, bloating, and mood swings in the subjects in relation to that normally observed with conventional HRT therapy. When used during days 1 through 14 to supplement the normal cycle for pre-menopausal subjects, the pulsed dosing regimen would be expected to provide the desired effects including the reduction or elimination of cycle bleeding, reduced hot flashes, and improved sleep.

To provide a clear and more consistent understanding of the specification and claims of this application, the following definitions are provided.

An alcohol-soluble species is a species that is insoluble in water and has a greater solubility in ethanol than in medium chain triglyceride (MCT) oils. For example, the nonderivatized hormone DHEA is soluble in ethanol up to approximately 150 mg/mL, thus being freely soluble, while having a solubility in MCT oil of only up to approximately 10 mg/mL, thus being only sparingly soluble. Alcohol-soluble species are preferably pharmacologically active, more preferably are a drug or a supplement, and neither include nor are water. Thus, liquids and solids may exist that technically are soluble in alcohol, but because they also are soluble in water or more or equivalently soluble in MCT oils than in ethanol are not “alcohol-soluble species”.

Nonderivatized hormones are chemically identical to hormones made by the human body and are not synthetically modified with fatty esters or other pendant groups.

Directly solubilize the nonderivatized hormone means that unlike in conventional systems, the nonderivatized hormone does not require synthetic conversion to an esterified state to be solubilized, thus the microemulsion “directly solubilizes” the nonderivatized hormone.

Phosphatidylcholine (PC) molecules are a subset of the larger set of phospholipids and are commonly used to form liposomes in water. When placed in water without other constituents, PC forms liposomes. In the presence of an oil, the application of sufficient shear forces to the PC liposomes in water can produce monolayer structures, including micelles. PC has a head that is water-soluble and a tail that is much less water-soluble in relation to the head. PC is a neutral lipid, but carries an electric dipole moment of about 10 D between the head and the tail, making the molecule itself polar.

Tocopheryl polyethylene glycol succinate 1000 (TPGS) is generally considered a surfactant having a non-polar, oil-soluble “Vitamin E” tail and a polar, water-soluble polyethylene glycol head. TPGS is a member of the polyethylene glycol derivatives that also include polysorbate 20, 40, 60, and 80.

MCT oils are triglycerides whose fatty acids have an aliphatic tail of 6-12 carbon atoms.

Room temperature and pressure means from 20 to 27 degrees Celsius at approximately 100 kPa.

Solid means a substance that is not a liquid or a gas at room temperature and pressure. A solid substance may have one of a variety of forms, including a monolithic solid, a powder, a gel, or a paste.

Liquid means as substance that is not a solid or a gas at room temperature and pressure. A liquid is an incompressible substance that flows to take on the shape of its container.

Solutions lack an identifiable interface between the solubilized molecules and the solvent. In solutions, the solubilized molecules are in direct contact with the solvent.

Solubilized means that the alcohol-soluble species to be delivered is in the solution of the droplet. When solubilized, dissociation (thus, liquid separation or solid formation) of the alcohol-soluble species does not result in droplet average particle diameters greater than 200 nm as determined by DLS and discussed further below, or by the formation of precipitated crystals of the alcohol-soluble species visible with the naked eye. Thus, if either average particle diameters greater than 200 nm or precipitated crystals visible to the naked eye form, the alcohol-soluble species is not solubilized in the solution of the droplet. If an alcohol-soluble species is not solubilized in the solution, it is insoluble in the solution. In many respects, solubility may be thought of as a concentration dependent continuum. For example, the following descriptive terms may be used to express solubility of a solute in a solvent (grams solid/mL of solvent) at 25 degrees Celsius:

| | Parts solvent per 1 part of |
|---|---|
| Descriptive Level | solute |
| Very Soluble | Less than 1 |
| Freely Soluble | From 1 to 10 |
| Soluble | From 10 to 30 |
| Sparingly Soluble | From 30 to 100 |
| Slightly Soluble | From 100 to 1000 |
| Very Slightly Soluble | From 1000 to 10,000 |
| Insoluble | More than 10,000 |

Dissociation occurs when a previously solubilized solid or liquid leaves a solution and is no longer in direct contact with a solvent of the solution. Dissociation of solids from the solvent occurs through recrystallization, precipitation, and the like. Dissociation of liquids from the solvent occurs through separation and the formation of a visible meniscus between the solvent and the dissociated liquid.

A shelf-stable microemulsion may be determined in one of two ways. One way to establish that a microemulsion stored in a sealed container substantially excluding air and moisture is shelf-stable is when dissociation of a solid does not occur and the oil phase droplets in the water do not change in average diameter by more than +/−20% at about 25° C. for a time period of at least 3 months to 2 years, preferably for a time period of at least 6 months to 2 years, and more preferably, for a time period of at least 1 year to 2 years. Another way to establish that a microemulsion is shelf-stable is when dissociation of a solid does not occur and the oil phase droplets in the water do not separate into a visibly distinct phase with a visible meniscus when stored in a sealed container substantially excluding air and moisture at about 25° C. for a time period of at least 6 months to 2 years, and more preferably, for a time period of at least 1 year to 2 years. Either type of dissociation means that the microemulsion is not shelf-stable.

Emulsions are mixtures of two or more liquids that do not solubilize. Thus, one of the liquids carries droplets of the second liquid. The droplets of the second liquid may be said to be dispersed in a continuous phase of the first liquid. An interface, separation, or boundary layer exists between the carrier liquid (continuous phase) and the droplets of the second liquid. Emulsions may be macroemulsions, pseudo-emulsions, microemulsions, or nanoemulsions. The primary difference between the emulsion types is the size (average diameter) of the droplets dispersed in the continuous phase and whether the droplets are thermodynamically stable in the continuous phase. Macroemulsions and pseudo-emulsions have average droplet diameters from 1 to 20 micrometers. Microemulsions and nanoemulsions have smaller average droplet diameters in the continuous phase than macroemulsions and pseudo-emulsions. Microemulsions are thermodynamically stable while nanoemulsions are not even though their average droplet diameters may overlap in size.

Macroemulsions are thermodynamically unstable but kinetically stable dispersions of oil in water, with oil being defined as any water-insoluble liquid. By thermodynamically unstable it is meant that once created, the macroemulsion is always reverting to the original, immiscible state of the oil and water constituents (demulsification), but this break down is slow enough (thus, kinetically “stable”) that the macroemulsion may be considered stable from an intended use practicality perspective. Macroemulsions scatter light effectively and therefore appear milky because their droplets are greater in diameter than the wavelength of visible light. The IUPAC definition of a macroemulsion is an “emulsion in which the particles of the dispersed phase have diameters from approximately 1 to 100 micrometers. Macro-emulsions comprise large droplets and thus are ‘unstable’ in the sense that the droplets sediment or float, depending on the densities of the dispersed phase and dispersion medium.”

Pseudo-emulsions are dispersions of oil in water, with oil being defined as any water-insoluble liquid, including tiny (micronized) solid granules that are not fully solubilized in the oil droplets. The term “pseudo-emulsion” is used as these mixtures are not true emulsions as the solid granules are not fully solubilized into the droplets. The droplets of a pseudo-emulsion generally have an average droplet diameter of 1 to 20 micrometers, thus being a “solid granule modified macroemulsion”.

Microemulsions are thermodynamically stable dispersions of oil in water, with oil being defined as any water-insoluble liquid. Microemulsion are made by simple mixing of the components. Thus, microemulsions spontaneously form or “self-assemble” and do not require high-energy forces to form. Unlike macroemulsions, microemulsions do not substantially scatter light. The IUPAC definition of a microemulsion is a “dispersion made of water, oil, and surfactant(s) that is an isotropic and thermodynamically stable system with dispersed domain diameter varying approximately from 1 to 100 nm, usually 10 to 50 nm.” Thus, the droplets of a microemulsion are approximately three orders of magnitude smaller than the droplets of a macroemulsion and are thermodynamically stable.

Nanoemulsions have average droplet diameters from 10 to 125 nanometers, thus being at least an order of magnitude smaller in average droplet diameters than macro- and pseudo-emulsions. Nanoemulsions are made with mechanical, high-energy forces-such as provided by high-pressure homogenization, high-shear mixers, such as bead mills and rotor-stator mixers, and ultrasonic mixers. While the average droplet diameter of nanoemulsions and microemulsions formally overlap, in practice, the average droplet diameter of nanoemulsions is or becomes larger than those of microemulsions, as lacking the thermodynamic stability of microemulsions, the average droplet diameter of nanoemulsions is forever increasing. It is possible to apply the high energy forces required to form a nanoemulsion to a composition capable of forming a thermodynamically stable microemulsion, however, this will result in a microemulsion as the composition would have “self-assembled” without the high energy forces to form the microemulsion.

Droplets or liquid particles are formed by the hydrophobic “oil” phase of a microemulsion and are carried by the hydrophilic continuous phase. The exterior of the droplets is defined by a boundary layer that surrounds the volume of each liquid droplet. The boundary layer of a droplet defines the exterior surface of the droplets forming the dispersed oil phase of the microemulsion. The continuous phase of the microemulsion resides exterior to the boundary layer of the droplets, and thus, carries the droplets.

Continuous phase means the portion of a microemulsion that carries the droplets that include the substance to be delivered. For example, the modified oil-in-water microemulsions (non-polar droplets in a polar continuous phase) addressed herein have oil/alcohol droplets including the alcohol-soluble species to be delivered carried in a polar, “water” continuous phase. While the words “water” and “oil” are used, the “water” can be any liquid that is more polar than the “oil” (such as a polar oil), and the “oil” can be any liquid that is less polar than the “water. Thus, the terms “polar continuous phase” and “water continuous phase” are synonymous, unless water is specifically being discussed as one of the microemulsion components.

Average droplet diameter is determined by dynamic light scattering, sometimes referred to as photon correlation spectroscopy. The determination is made between 20 and 25 degrees Celsius. One example of an instrument suitable for average droplet diameter determination is a Nicomp 380 ZLS particle sizer as available from Particle Sizing Systems, Port Richey, FL. DLS can determine the diameter of droplets in a liquid by measuring the intensity of light scattered from the droplets to a detector over time. As the droplets move due to Brownian motion the light scattered from two or more droplets constructively or destructively interferes at the detector. By calculating the autocorrelation function of the light intensity and assuming a droplet distribution, it is possible to determine the sizes of droplets from 1 nanometer to 5 micrometers. The instrument is also capable of measuring the Zeta potential of droplets.

A visually clear microemulsion has an average droplet diameter of 200 nm and less and lacks precipitated solid crystals visible to the naked eye.

A transparent microemulsion or nanoemulsion has an average droplet diameter from 10 to 100 nanometers. Thus, a transparent microemulsion or nanoemulsion is visually clear, but a visually clear microemulsion or nanoemulsion may or may not also be transparent.

Microemulsion viscosity is determined using a Gilmont Falling Ball Viscometer with a number three (#3) tube size equipped with a tantalum ball. Such viscometers may be obtained, for example, from COLE-PARMER®), 625 East Bunker Ct Vernon Hills, IL. Viscosity determinations were made at 19 degrees Celsius using an approximately 7 mL sample. The tantalum ball density was 16.6 g/mL and the viscometer constant for tube #3 was 35. The equation used to determine viscosity was μ=K(ρt−ρ)t, where u is viscosity in cp, ρt is ball density, ρ is liquid density, and time is in minutes.

A micronized powder is a solid powder having an average particle diameter from 0.1 to 100 microns. Micronized powders of nonderivatized hormones generally have average particle diameters of 50 microns and less.

Ingestible means capable of being ingested through the mouth by a living mammal while edible means fit to be eaten, thus in contrast to being unpalatable or poisonous. Edible also means that the composition has less than the permitted amount of viable aerobic microorganisms and meets the American Herbal Products Association (AHPA) guidelines for metals, adulterants, toxins, residual solvents, and pesticides.

Subject refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. Thus, the term “subject” may be used in reference, for example, to a mammalian subject, such as a male or female human.

Therapeutically effective amount refers to the amount of a compound that, when administered, is sufficient to prevent development of, or alleviate to some extent, one or more of the symptoms of the disorder being addressed. The term “therapeutically effective amount” also refers to the amount of a compound that is sufficient to elicit the biological or medical response of a cell, tissue, system, animal, or human that is being sought by a researcher, veterinarian, medical doctor, or clinician.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these ranges may independently be included in the ranges, and are also encompassed within the invention, subject to any specifically excluded limit in the stated range. Where the stated range includes one or both of the upper and lower limits, ranges excluding either or both of those included limits are also included in the invention.

Unless otherwise indicated, all numbers expressing quantities of ingredients, ratios, and the like used in the specification and claims are to be understood as indicating both the exact values as shown and as being modified by the term “about”. Thus, unless indicated to the contrary, the numerical values of the specification and claims are approximations that may vary depending on the desired properties sought to be obtained and the margin of error in determining the values. At the very least, and not as an attempt to limit the application of the doctrine of equivalents to the scope of the claims, each numerical parameter should at least be construed considering the margin of error, the number of reported significant digits, and by applying ordinary rounding techniques.

The terms “a”, “an”, and “the” used in the specification claims are to be construed to cover both the singular and the plural, unless otherwise indicated or contradicted by context. No language in the specification should be construed as indicating any non-claimed element to be essential to the practice of the invention.

The terms “topical” and “transdermal” are used interchangeably to refer to a composition that when applied to the skin of a subject transfers a deliverable through the skin to the bloodstream of the subject. Outside of this application, one may find the word “topical” used to describe a composition that only transfers a deliverable to the skin and not to the bloodstream.

While various aspects of the invention are described, it will be apparent to those of ordinary skill in the art that other aspects and implementations are possible within the scope of the invention. Accordingly, the invention is not to be restricted except considering the attached claims and their equivalents.

1. A thickened modified oil-in-water microemulsion composition comprising:
an alcohol-soluble species;
an alcohol-lipid phase thickener, where the alcohol-lipid phase thickener is alcohol-soluble;
a continuous phase thickener, where the continuous phase thickener is water-soluble; and
a modified oil-in-water microemulsion comprising a modified oil phase and a modified polar continuous phase,
where the alcohol-soluble species is solubilized in the modified oil phase, the modified oil phase comprising a phospholipid, a polyethylene glycol derivative, an oil, and an alcohol, and
where the modified polar continuous phase comprises water.

2. The composition of claim 1, where the modified oil phase comprises the alcohol-lipid phase thickener and the modified polar continuous phase comprises the continuous phase thickener.

3. The composition of claim 1, where the modified oil phase and the modified polar continuous phase comprise the alcohol-lipid phase thickener and the modified polar continuous phase comprises the continuous phase thickener.

4. The composition of claim 1, where the modified polar continuous phase comprises the alcohol-lipid phase thickener and the continuous phase thickener.

5. The composition of claim 1, where a ratio of the alcohol-lipid phase thickener to the oil is 1:0.6±20% by weight.

6. The composition of claim 1, the alcohol-lipid phase thickener having significant water-solubility.

7. The composition of claim 6, where the alcohol-lipid phase thickener is chosen from alkyl benzoates, glycols, and combinations thereof.

8. The composition of claim 7 where the alcohol-lipid phase thickener is chosen from butylene glycol (1,3-butanediol), propylene glycol, hexylene glycol, ethoxydiglycol, di-propylene glycol, alkyl benzoate, and combinations thereof.

9. The composition of claim 6, where the alcohol-lipid phase thickener is butylene glycol.

10. The composition of claim 1, where the continuous phase thickener is chosen from salts of hyaluronic acid, xanthin gum, acacia gum, and combinations thereof.

11. The composition of claim 1 where the continuous phase thicker is a potassium salt of hyaluronic acid.

12. The composition of claim 1, where the thickened modified oil-in-water microemulsion is shelf-stable.

13. The composition of claim 1, where the thickened modified oil-in-water microemulsion is visually clear.

14. The composition of claim 1, where the thickened modified oil-in-water microemulsion is transparent.

15. The composition of claim 1, where the modified oil phase is dispersed in the modified polar continuous phase.

16. The composition of claim 15, where droplets of the modified oil phase have an average droplet diameter of 1 to 100 nanometers.

17. The composition of claim 15, where droplets of the modified oil phase have an average droplet diameter of 7 to 30 nanometers.

18. The composition of claim 1, where the alcohol-soluble species comprises a nonderivatized hormone.

19. The composition of claim 18, the nonderivatized hormone chosen from testosterone, dehydroepiandrosterone (3-beta-hydroxyandrosteron-5-en-17-one), dihydrotestosterone, 7-keto dehydroepiandrosterone, pregnenolone, androstenedione, androstenediol, progesterone, estradiol, estrone, estriol, cortisol, hydrocortisone, and combinations thereof.

20. The composition of claim 18, the nonderivatized hormone chosen from testosterone, progesterone, and dehydroepiandrosterone.

21. The composition of claim 18, where the nonderivatized hormone is progesterone.

22. The composition of claim 18, where the modified oil phase directly solubilizes the nonderivatized hormone.

23. The composition of claim 22, the modified oil phase further comprising a derivatized hormone.

24. The composition of claim 1, the modified oil phase further comprising an oil-soluble vitamin.

25. The composition of claim 24, where the oil-soluble vitamin is chosen from Vitamin A, Vitamin D, Vitamin E, Vitamins K1, Vitamin K2, and combinations thereof.

26. The composition of claim 1, where the phospholipid is a glycerophospholipid isolated from lecithin.

27. The composition of claim 26, where the glycerophospholipid isolated from lecithin is chosen from phosphatidylcholine, phosphatidylethanolamine, phosphatidylinositol, ceramide phosphoryl ethanolamine, ceramide phosphoryl choline (SPH), and combinations thereof.

28. The composition of claim 26, where the glycerophospholipid isolated from lecithin is chosen from phosphatidylcholine, phosphatidylethanolamine, and combinations thereof.

29. The composition of claim 26, where the glycerophospholipid isolated from lecithin is at least 80% by weight phosphatidylcholine.

30. The composition of claim 1, where the polyethylene glycol derivative is chosen from polyethylene glycol modified vitamin E, polysorbate 40, polysorbate 60, polysorbate 80, and combinations thereof.

31. The composition of claim 30, where the polyethylene glycol modified vitamin E is tocopheryl polyethylene glycol succinate 1000.

32. The composition of claim 1, the oil chosen from a medium chain triglyceride, a citrus oil, and combinations thereof.

33. The composition of claim 32, the medium chain triglyceride chosen from caproic acid (hexanoic acid), caprylic acid (octanoic acid), capric acid (decanoic acid), lauric acid (dodecanoic acid), and combinations thereof.

34. The composition of claim 32, the medium chain triglyceride chosen from caprylic acid (octanoic acid), capric acid (decanoic acid), and combinations thereof.

35. The composition of claim 32, the citrus oil chosen from orange oil, lemon oil, and combinations thereof.

36. The composition of claim 1, the modified polar continuous phase further comprising a water-soluble deliverable.

37. The composition of claim 36, the water-soluble deliverable chosen from Vitamin C, Vitamin B5, Nicotinamide MonoNucleotide (NMN), Nicotinamide Adenine Dinucleotide (NAD), Glutathione, nitric oxide, L-citrulline, L-carnosine, aloe, beta-Hydroxy beta-Methyl Butyric acid (HMB), and combinations thereof.

38. The composition of claim 1, where the alcohol is 95% ethanol by weight.

39. The composition of claim 1, where the alcohol-soluble species comprises from 0.2% to 5% of the composition by weight.

40. The composition of claim 1, where a ratio of the phospholipid, to the oil, to the polyethylene glycol derivative, to the alcohol, to the alcohol-lipid phase thickener, to the continuous phase thickener, and to the water is 1:1:0.6-3.3:1.8:1.8:0.02-2.5:7±20% by weight.

41. The composition of claim 1, where a ratio of the phospholipid, to the oil, to the polyethylene glycol derivative, to the alcohol, to the alcohol-lipid phase thickener, to the continuous phase thickener, and to the water is 1:1:0.6-3.3:1.8:1.8:0.02-2.5:7±10% by weight.

42. The composition of claim 1, where a ratio of the oil to the alcohol-soluble species is 1:0.02 to 1:0.3±10% by weight in the modified oil phase.

43. The composition of claim 1, where the phospholipid comprises from 3% to 10% of the composition by weight.

44. The composition of claim 1, where the polyethylene glycol derivative comprises from 5% to 14% of the composition by weight.

45. The composition of claim 1, where a ratio of the phospholipid to the polyethylene glycol derivative is 1:0.4 to 1:5 by weight.

46. The composition of claim 1, where a ratio of the phospholipid to the polyethylene glycol derivative is 1:0.8 to 1:5 by weight.

47. The composition of claim 1, where the oil comprises from 5% to 15% of the composition by weight.

48. The composition of claim 1, where the alcohol comprises from 5% to 25% of the composition by weight.

49. The composition of claim 1, where a ratio of the oil to the alcohol is 1:0.8 to 1:3 by weight.

50. The composition of claim 1, where the alcohol-lipid phase thickener comprises from 8% to 16% of the composition by weight.

51. The composition of claim 1, where the alcohol-lipid phase thickener is butylene glycol.

52. The composition of claim 1, where the continuous phase thickener comprises from 0.15% to 5% of the composition by weight.

53. The composition of claim 1, where a ratio of the continuous phase thickener to the alcohol-lipid phase thickener is from 1:30 to 1:50 by weight.

54. The composition of claim 1, where the water comprises from 47% to 55% of the composition by weight.

55. The composition of claim 1, where the thickened modified oil-in-water microemulsion is configured to provide a skin residence time of 3 to 8 minutes.

56. The composition of claim 55, where the skin residence time of 3 to 8 minutes significantly reduces the ability to transfer the composition to surfaces other than where initially applied.

57. The composition of claim 1, where the thickened modified oil-in-water microemulsion is configured to provide transdermal uptake of the alcohol-soluble species to the bloodstream of a subject at a therapeutically effective bloodstream concentration.

58. The composition of claim 1, where the alcohol-soluble species is progesterone, and the composition is configured to provide a human subject a 2 to 8 ng/ml blood concentration increase of the progesterone or a metabolite of the progesterone over a baseline bloodstream concentration within 90-minutes of applying the composition to skin of the human subject, where the composition comprises approximately 20 mg of the progesterone.

59. The composition of claim 1, where the alcohol-soluble species is progesterone, and the composition is configured to provide a human subject at least twice the progesterone bloodstream concentration than the progesterone bloodstream concentration provided by a cream comprising the same amount of the progesterone as the composition within two hours of application to the skin of the human subject, where the cream lacks the thickened modified oil-in-water microemulsion.

60. The composition of claim 1, where the alcohol-soluble species is progesterone, and the composition is configured to provide a human subject at least six times the progesterone bloodstream concentration than the progesterone bloodstream concentration provided by a cream comprising the same amount of the progesterone as the composition within six hours of application to the skin of the human subject, where the cream lacks the thickened modified oil-in-water microemulsion.

61. The composition of claim 1, where the alcohol-soluble species is progesterone, and the composition is configured to provide a higher percentage of the progesterone applied to the skin of a human subject to the bloodstream of the human subject in relation to a cream comprising the progesterone, where the cream lacks the thickened modified oil-in-water microemulsion.

62. The composition of claim 1, where the alcohol-soluble species is progesterone, and the composition is configured to deliver from 3 to 6 times more of the progesterone to the bloodstream of a human subject on a cumulative basis two hours after application to the skin than a cream comprising the same amount of the progesterone as the composition, where the cream lacks the thickened modified oil-in-water microemulsion.

63. The composition of claim 1, where the alcohol-soluble species is progesterone, and the composition is configured to deliver from 12 to 17 times more of the progesterone to the bloodstream of a human subject on a cumulative basis six hours after application to the skin than a cream comprising the same amount of the progesterone as the composition, where the cream lacks the thickened modified oil-in-water microemulsion.

64. A method of making a thickened modified oil-in-water microemulsion composition, the method comprising:
combining a phospholipid, a polyethylene glycol derivative, an oil, an alcohol, and an alcohol-lipid phase thickener to form an alcohol-lipid mixture;
combining a continuous phase thickener and water to form a modified polar continuous phase; and
combining an alcohol-soluble species with the alcohol-lipid mixture and the modified polar continuous phase at atmospheric pressure to form the thickened modified oil-in-water microemulsion.

65.-70. (canceled)

71. A method of delivering an alcohol-soluble species to the bloodstream of a human subject, the method comprising:
applying a thickened modified oil-in-water microemulsion composition to skin of a human subject, where the composition comprises:
an alcohol-soluble species;
an alcohol-lipid phase thickener, where the alcohol-lipid phase thickener is alcohol-soluble;
a continuous phase thickener, where the continuous phase thickener is water-soluble; and
a modified oil-in-water microemulsion comprising a modified oil phase and a modified polar continuous phase,
where the alcohol-soluble species is solubilized in the modified oil phase, the modified oil phase comprising a phospholipid, a polyethylene glycol derivative, an oil, and an alcohol, and
where the modified polar continuous phase comprises water; and
delivering the alcohol-soluble species to the bloodstream of the human subject,
where within 90-minutes of the applying the composition to the skin of the human subject, approximately 1 mL of the composition provides the human subject a blood concentration from 2 to 8 ng/ml of the alcohol-soluble species or a metabolite of the alcohol-soluble species over a baseline pre-application bloodstream concentration of the alcohol-soluble species.

72.-79. (canceled)

80. A method of treating a female human subject in need of progesterone replacement therapy with a pulsed progesterone dosage regimen, the method comprising:
applying a thickened modified oil-in-water microemulsion composition comprising a therapeutically effective amount of progesterone for a treatment duration of at least two weeks,
where the applying occurs once daily to the skin, and
where the composition comprises:
an alcohol-soluble species;
an alcohol-lipid phase thickener, where the alcohol-lipid phase thickener is alcohol-soluble;
a continuous phase thickener, where the continuous phase thickener is water-soluble; and
a modified oil-in-water microemulsion comprising a modified oil phase and a modified polar continuous phase,
where the alcohol-soluble species is solubilized in the modified oil phase, the modified oil phase comprising a phospholipid, a polyethylene glycol derivative, an oil, and an alcohol, and
where the modified polar continuous phase comprises water;
exceeding at least a baseline bloodstream concentration of progesterone of the human subject within 90-minutes of the applying to produce an elevated progesterone blood concentration in the bloodstream of the human subject;
reducing the elevated progesterone concentration in the bloodstream of the human subject to approximate the baseline bloodstream concentration in the bloodstream of the human subject within eight hours of the applying; and
reducing breast tenderness, bloating, and mood swings in the human subject in relation to the breast tenderness, bloating, and mood swings that would occur when the total amount of progesterone applied over the treatment period is introduced to the skin as a cream lacking the modified oil-in-water microemulsion.

81.-86. (canceled)
